(19) United States
(12) Patent Application Publication
(10) Pub. No.: US 2024/0052412 A1
(43) Pub. Date: Feb. 15, 2024

(54) METHOD FOR DETECTING RNA STRUCTURE AT WHOLE TRANSCRIPTOME LEVEL AND USE THEREOF

(71) Applicant: TSINGHUA UNIVERSITY, Beijing (CN)

(72) Inventors: Qiangfeng ZHANG, Beijing (CN); Meiling PIAO, Beijing (CN)

(21) Appl. No.: 18/260,438

(22) PCT Filed: Nov. 5, 2020
(86) PCT No.: PCT/CN2020/126766

§ 371 Date: Jul. 5, 2023

(51) Int. Cl. C12Q 1/6869; C12Q 1/6855

(52) U.S. Cl. CPC C12Q 1/6869; C12Q 1/6855; C12Q 1/6806

(57) ABSTRACT

Provided in the present invention are a method for detecting an RNA structure and the use thereof. According to the present invention, the step of removing the background of reverse transcription termination signals is included in the method for detecting an RNA structure, and false positive signals in a structural score calculation are reduced, and therefore the accuracy of the detection method is improved.

### TECHNICAL FIELD

The present invention belongs to the technical field of biology, and particularly relates to a whole transcriptome level RNA structure probing method and use thereof.

### BACKGROUND

RNA has different functions, for example: as messengers to convey genetic information, or as ribozymes to catalyze reactions. RNA molecules are precisely regulated throughout their entire life cycle and at different subcellular locations. The complex and flexible structures are the core of the functional diversity and fine regulation of RNA molecules. Misfolding of RNA structures can interfere with processes such as alternative splicing, translation, RNA modification and editing, and RNA-protein interactions, thereby leading to disease.

RNA structure probing methods utilize chemical reagents that specifically modify single-stranded nucleotides. The modification sites can interfere with reverse transcription (RT), resulting in RT stops or mutations; therefore, the modification sites can be detected by sequencing and bioinformatic analyses, and RNA structural information is thus obtained. Most reagents can only probe structural information of one or two bases; for example, dimethyl sulfate (DMS) modifies single-stranded cytosines and adenines, glyoxal modifies single-stranded guanines, cytosines and adenines, and kethoxal modifies single-stranded guanines. Selective 2-hydroxy acylation analyzed by primer extension (SHAPE) reagents can modify the 2′ OH group of ribose within single-stranded regions and provide structural information for all four nucleotides.

Global RNA structure probing studies have revealed that structural differences often exist at functional RNA sites, such as protein and miRNA binding sites, and studies have shown that RNA structures can be involved in regulating the splicing, translation and degradation processes of RNA. Notably, several studies have shown that RNA sequences can form different structures in vivo and in vitro, at different subcellular compartments, and at different stages of embryogenesis. Indeed, many factors in cells can affect RNA structures, including pH, cation concentrations, endogenous RNA modifications (e.g., methylation, acetylation), and interactions with proteins and/or other RNAs. Therefore, studying RNA structures in their most relevant natural environments is crucial for revealing RNA functions and regulatory mechanisms.

However, current state-of-the-art RNA structure probing methods typically require a large amount of RNA as input, which limits their practical uses. For example, the construction of RNA libraries for icSHAPE and Structure-seq2 requires approximately 10⁷ cells, which is difficult to achieve for biological studies of rare primary cells and many tissue samples. Therefore, in addition to some studies on zebrafish early embryos and drosophila ovaries that are experimentally easy to collect, RNA structure probing studies are as yet limited to cultured cell lines. However, the cellular environments in cell lines and the RNA structures generated therefrom may deviate significantly from the primary sample, such that the results cannot truly reflect the functional states of the cells.

### SUMMARY

To overcome this obstacle, we developed smartSHAPE (small amount random RT icSHAPE), a novel secondary structure probing method for low amounts of input RNA, which is an improvement over the icSHAPE method. Therefore,

In a first aspect of the present invention, an RNA structure probing method is provided, wherein the method comprises:

1. obtaining an RNA-containing sample; 2. preparing a smartSHAPE library; and 3. RNA structure probing and analysis, wherein in step 2, preparing the smartSHAPE library comprises: (1) RNA modification and preparation; (2) RNA reverse transcription, removal of background reverse transcription stop signals caused by non-modification sites (premature RT stops), and cDNA enrichment.

Preferably, step 2 of the RNA structure probing method further comprises (3) adapter ligation, second strand synthesis, and amplification. More preferably, the adapter ligation includes 3′ adapter ligation and 5′ adapter ligation.

Preferably, the background reverse transcription stop signals are caused by non-RNA modification sites. More preferably, the background reverse transcription stop signals may be derived from endogenous modifications (e.g., m¹A modifications), local structures (e.g., G-quadruplexes), or random shedding of reverse transcriptase.

More preferably, the background reverse transcription stop signals are removed by ribonuclease (RNase) digestion. More preferably, the background reverse transcription stop signals are removed by RNase I digestion.

Preferably, a primer for the reverse transcription (RT) has the sequence of 5′-NNNNNN-3′, 5′-NNWNNWNN-3′, or 5′-TTTTTTTTVN-3′. Preferably, the RNA is modified with a labeling reagent; more preferably, the labeling reagent is a cell membrane penetrating reagent; more preferably, the labeling reagent is dimethyl sulfate (DMS), 1-methyl-7-nitroisatoic anhydride (1M7), 2-methylnicotinic acid imidazolide-azide (NAI-N3) or kethoxal; more preferably, the labeling reagent is 2-methylnicotinic acid imidazolide-azide (NAI-N3).

Preferably, the cDNA enrichment is enrichment with magnetic beads; more preferably, the magnetic beads are streptavidin magnetic beads, such as MyOne C1 magnetic beads.

Preferably, the RNA structure is an RNA secondary structure.

Preferably, the RNA is a full-length RNA; further, the RNA is a transcriptome RNA. It may be a long-chain RNA, such as an mRNA, lncRNA or rRNA, or it may comprise many small RNAs, such as small RNAs smaller than 200 nt, protein-bound RNAs, or RNAs serving as Dicer substrates.

Preferably, the RNA may be derived from any cell, virus, etc.; preferably, the cell includes, but is not limited to, cell lines cultured in laboratories, living cells, primary cells, mammalian early embryos, bacteria, fungi, and various infected cells, such as cells infected by viruses, bacteria, fungi, etc.; more preferably, the living cells may be any somatic cell or germ cell, such as epithelial cells, dermal cells, glandular cells, blood-derived cells, bone cells, immune cells (T cells, B cells, NK cells, macrophages, etc.), or fertilized eggs.

The RNA structure probing method further comprises a processing step of calculating smartSHAPE scores using a computational pipeline. The calculation processing step comprises: 1) removing a 3′ adapter; 2) removing duplicate reads; 3) removing a molecular label; 4) aligning clean reads to rRNA standard sequences; 5) aligning reads that are not aligned to rRNA sequences to a genome; 6) converting Sam files into .tab files using icSHAPE-pipe sam2tab; and 7) calculating smartSHAPE scores using icSHAPE-pipe calcSHAPENoCont.

Preferably, in step 7), the smartSHAPE scores are calculated by normalization and winsorization of RT stop counts across all exons in a sliding window fashion, and the scores for bases with coverage below 100 are defined as NULL.

More preferably, parameters in step 7) are: —N NAI_rep1.tab, NAI_rep2.tab; -size chrNameLength.txt; -out reactivity.gTab; -ijf sjdbList.fromGTF.out.tab.

Preferably, the probing method does not comprise a gel recovery step before library amplification.

Preferably, in the library construction of the computational pipeline, no control group is required to remove background signals.

Preferably, in the RNA structure probing method, RNA structure probing can be performed with an RNA input of as little as 1 ng (10⁴ to 10⁵ cells).

The present invention further provides use of the RNA structure probing method described above, the use comprising assessing functional states of cells, studying the effect of RNA on early development and the development and progression of cancer, etc., according to the result of the probing method described above.

Preferably, the functional states include various physiological and abnormal states, such as cellular inflammation, injury, ischemia, immune stress state, early developmental process, infection, and cancer proliferation. More preferably, the infection is caused by viruses, bacteria, fungi, etc.

Preferably, the cells are derived from any tissue organ, such as the cutaneous system, the blood lymphatic system, the immune system, the cardiovascular system, the digestive system, the respiratory system, the urinary system, the skeletal system, the reproductive system, or the nervous system.

Preferably, the cells include immune cells, such as B cells, T cells, NK cells, and macrophages.

Preferably, the use is not a diagnosis or treatment method for a disease.

The present invention further provides a method for assessing a functional state of a cell, the assessing method comprising probing RNA structures of the cell by any probing method described above, and assessing the functional state of the cell according to the probing result.

Preferably, the functional state of the cell is cellular inflammation, injury, ischemia, immune stress state, early developmental process, infection, cancer proliferation, etc.; more preferably, the infection is caused by viruses, bacteria, fungi, etc.

More preferably, the functional state of the cell is an immune stress state of the cell. An example is an immune stress state of an immune cell. Still further preferably, the immune cell includes, for example, B cells, T cells, NK cells, and macrophages.

The present invention has the following beneficial technical effects:

1. The present invention removes the background reverse transcription stop signals, reducing false positive signals caused by the background reverse transcription stop signals in the structure score calculation, thereby improving the accuracy of the probing method.

2. The present invention adopts a different library construction strategy, wherein we combine random RT with on-bead single-stranded DNA library construction, greatly reducing the losses caused by multiple purification steps.

3. SmartSHAPE requires an RNA input of as little as 1 ng (10⁴ to 10⁵ cells), enabling RNA structure analysis of in vivo cells at a very low sample amount. The method can be applied to any cell, such as rare primary cells, mammalian early embryos, and patient biopsy samples.

4. We used smartSHAPE to describe the whole transcriptome RNA secondary structure of intestinal macrophages from bacterial infection model mice, wherein only 100 ng of total RNA was used as input for each sample. We revealed differences in RNA structure between two populations of macrophages after immune stress, which are rich in immune response-associated genes, and we provided evidence for regulation of immune response through RNA structure.

5. The smartSHAPE of the present invention is an efficient, accurate and robust method for studying whole transcriptome RNA secondary structures in vivo that requires only a very small amount of RNA as input. Our method integrates random reverse transcription, RNase I digestion, and on-bead library construction to increase the efficiency of library construction and to generate accurate RNA structural data. The results of the present invention show that smartSHAPE successfully removes background reverse transcription stop signals by RNase I digestion followed by magnetic bead enrichment, and achieves better accuracy than icSHAPE even without a DMSO group as a control.

6. In view of the minimal requirements of the method of the present invention for RNA initial material, it is very promising to apply smartSHAPE to the study of the widespread roles of RNA structure in many other potential biological environments. For example, maternal RNA degradation is essential for early development, and several studies have reported that RNA structure plays a regulatory role in maternal RNA degradation during early embryogenesis of zebrafish. The RNA structurome in mammalian early embryos has not been studied due to the limited sample amount in the prior art, but can be approached by smartSHAPE of the present invention. In addition, dysregulation of RBP binding is known to be involved in the development and progression of many cancers. SmartSHAPE may provide a viable means to study these dysregulations from the perspective of RNA structure by using rare biopsy samples from the clinic. In addition, when used in combination with enrichment (e.g., by antisense oligonucleotides or protein antibodies), smartSHAPE is expected to help discover and functionally validate regulatory effects based on RNA structure; these RNAs include RNAs expressed at low levels (such as many lncRNAs), RNA species in stress granules, and RNA fragments bound by RBPs, etc.

The foregoing is merely a summary of some aspects of the present invention, and is not, and should not be construed as, limiting the present invention in any way.

Unless otherwise specified, the practice of the present invention will adopt traditional techniques of cell biology, cell culture, molecular biology, immunology, and the like. These techniques are explained in detail in the following documents. For example:
1. Xu, H. et al. Notch-RBP-J signaling regulates the transcription factor IRF8 to promote inflammatory macrophage polarization. Nat Immunol 13, 642-650, doi:10.1038/ni.2304 (2012);
2. Li, P., Shi, R. & Zhang, Q. C. icSHAPE-pipe: A comprehensive toolkit for icSHAPE data analysis and evaluation. Methods 178, 96-103, doi:10.1016/j.ymeth.2019.09.020 (2020);
3. Bolger, A. M., Lohse, M. & Usadel, B. Trimmomatic: a flexible trimmer for Illumina sequence data. Bioinformatics 30, 2114-2120, doi:10.1093/bioinformatics/btul70 (2014);
4. Langmead, B. & Salzberg, S. L. Fast gapped-read alignment with Bowtie 2. Nat Methods 9, 357-359, doi:10.1038/nmeth.1923 (2012);
5. Dobin, A. et al. STAR: ultrafast universal RNA-seq aligner. Bioinformatics 29, 15-21, doi:10.1093/bioinformatics/bts635 (2013);
6. Pedregosa, F. et al. Scikit-learn: Machine Learning in Python. J Mach Learn Res 12, 2825-2830 (2011);
7. Reuter, J. S. & Mathews, D. H. RNA structure: software for RNA secondary structure prediction and analysis. BMC Bioinformatics 11, 129, doi:10.1186/1471-2105-11-129 (2010);
8. Spitale, R. C. et al. Structural imprints in vivo decode RNA regulatory mechanisms. Nature 519, 486-490, doi:10.1038/nature14263 (2015).

All patents and publications mentioned in this specification are herein incorporated by reference in their entirety. Those skilled in the art should recognize that certain changes may be made to the present invention without departing from the conception or scope of the present invention. The following examples further illustrate the present invention in detail and should not be construed as limiting the scope of the present invention or the specific methods described herein.

### BRIEF DESCRIPTION OF THE DRAWINGS

FIG. 1: a schematic diagram of smartSHAPE library preparation;

FIG. 2: optimization of RNA fragmentation and 3′ DNA adapter ligation steps, wherein FIG. 2a shows the yield and fragment distribution of NAI-N3 modified or unmodified HEK293T total RNA under different fragmentation conditions; FIG. 2b is a schematic diagram of adapters of three different structures, including a short adapter, a long adapter comprising a 10-base barcode, and an adapter formed by adding a random nucleotide to the 5′ end of the long adapter; FIG. 2c shows products of ligation of an adapter to the 3′ end of a synthesized DNA molecule with CircLigase and T4 DNA Ligase.

FIG. 3: removal of background noise by RNase I digestion in smartSHAPE, wherein FIG. 3a is a schematic diagram of background noise removal by RNase I digestion and magnetic bead enrichment; FIG. 3b shows the site of a known m¹A modification in 28S ribosomal RNA; FIG. 3c shows a primer designed upstream of the m¹A site, and background reverse transcription signal detection; FIG. 3d shows the difference in reverse transcription stop signals between the DMSO group and the NAI-N3 group at the known m¹A modification site of endogenous m¹A or m³U;

FIG. 3e shows a sequence of 18S ribosomal RNA, with the smartSHAPE values calculated with the NAI-N3 group only shown on the left and the icSHAPE values calculated with the NAI-N3 group and the DMSO group shown on the right; FIG. 3f shows ROC curves corresponding to two SHAPE values calculated for 18S ribosomal RNA.

FIG. 4: RNase I digestion can effectively remove background signals, wherein FIG. 4a shows a synthesized RNA sequence and a structure; FIG. 4b shows the background reverse transcription signals caused by removal of m¹A modifications, when RNase I digestion and magnetic bead enrichment are simultaneously performed on the product of reverse transcription following NAI-N3 modification of two synthesized RNAs which have been separately folded in vitro; FIG. 4c shows a library construction process for the DMSO group; FIG. 4d shows the difference distribution of reverse transcription stop signals of the DMSO group and the NAI-N3 group for all ribosomal RNA sites, with the different lines representing the mean values of stop signal differences for all known endogenous modification sites in the ribosomal RNA; FIG. 4e is the distribution of reverse transcription stop signals in different NAI-N3 libraries at sites with abnormally high background signals.

FIG. 5: the coverage and accuracy of smartSHAPE with different RNA inputs, wherein FIG. 5a shows reverse transcription stop signals at each site of the RPS16 transcripts for smartSHAPE and icSHAPE libraries of four different inputs; FIG. 5b shows the number of transcripts with high coverage for smartSHAPE and icSHAPE libraries of four different RNA inputs under different sequencing depths; FIG. 5c shows the number of reads corresponding to each processing step for smartSHAPE and icSHAPE libraries of four different RNA inputs; FIG. 5d shows the ROC curves of smartSHAPE and icSHAPE libraries of four different RNA inputs in 18S and 28S ribosomal RNAs; FIG. 5e shows AUCs of smartSHAPE and icSHAPE libraries of four different RNA inputs at XBP1 structure element, corresponding to SHAPE scores at the site.

FIG. 6: smartSHAPE libraries of different inputs show high reproducibility and library complexity, wherein FIG. 6a shows the correlation of SHAPE scores of smartSHAPE and icSHAPE libraries of four different inputs (1 ng, 5 ng, 25 ng, and 125 ng); FIG. 6b shows the distribution of Pearson correlation between different library technology replicates for sites having SHAPE scores in each transcript of smartSHAPE and icSHAPE libraries of four different inputs (1 ng, 5 ng, 25 ng, and 125 ng); FIG. 6c shows the cumulative distribution curve of the average reverse transcription stop signals for each transcript in smartSHAPE libraries of four different inputs under different sequencing depths.

FIG. 7: the smartSHAPE library has similar probed structural features as icSHAPE, wherein FIG. 7a shows the average SHAPE value at each site in the interval from 30 bases upstream to 100 bases downstream of the start codon and in the interval from 100 bases upstream to 30 bases downstream of the stop codon for smartSHAPE and icSHAPE libraries; FIG. 7b shows the distribution of SHAPE scores of the four different bases A, U, G, and C in smartSHAPE and icSHAPE libraries of four different RNA inputs; FIG. 7c shows the average SHAPE score at each site around the m⁶A modification for smartSHAPE and icSHAPE libraries; FIG. 7d shows the distribution of the Gini index of different RNA species or regions in smartSHAPE and icSHAPE libraries.

FIG. 8: smartSHAPE is used to probe RNA structures of intestinal macrophages in a mouse, wherein FIG. 8a shows a flow chart of mouse macrophage separation and RNA secondary structure probing; FIG. 8b shows the number of transcripts with high coverage in smartSHAPE libraries of two types of macrophages, i.e., the number of transcripts with more than a coverage of 100 at more than 80% of sites; FIG. 8c shows AUCs of smartSHAPE and icSHAPE libraries of two types of macrophages at Xbp1 known structure element.

FIG. 9: Ly6C^{lo} tissue-resident macrophages and Ly6C^{hi} pro-inflammatory macrophages are sorted by flow cytometry based on the immune-related genes MHCII, CD45, SiglecF, CD11b, CD11c, CD64, and Ly6C.

FIG. 10: the accuracy of macrophage smartSHAPE data, wherein FIG. 10a shows AUCs of smartSHAPE and icSHAPE libraries of two types of macrophages for SRP RNA; FIG. 10b shows ROC curves and their respective area under the curve, which are generated, for each of 60 known RNA structures in the Rfam database, from smartSHAPE data of two types of macrophages and icSHAPE data of mouse embryonic stem cells, and shows the distribution of AUCs for each library.

### DETAILED DESCRIPTION

The present invention is further described with reference to the following specific examples, and the advantages and features of the present invention will be clearer as the description proceeds. These examples are illustrative only and do not limit the scope of the present invention in any way. It should be understood by those skilled in the art that modifications and replacements can be made to the details and form of the technical solutions of the present invention without departing from the spirit and scope of the present invention and that all these modifications and replacements fall within the scope of the present invention.

### Example 1: Whole Transcriptome Level RNA Structure Probing Method

In icSHAPE, NAI-N3 was used to modify RNAs in vivo in single-stranded regions. The RNAs were then fragmented, ligated to a 3′ adapter, and converted into double-stranded DNA libraries by reverse transcription, circligation, and amplification. Notably, icSHAPE library construction employs multiple steps of gel extraction and column purification steps, which lead to RNA sample loss, making it difficult or impossible to analyze samples with a small amount of input RNA. Even with a high recovery rate of 80% and 50% for column and gel purification, respectively, we typically obtained only a 5% yield after seven column purification steps and two gel size selection steps.

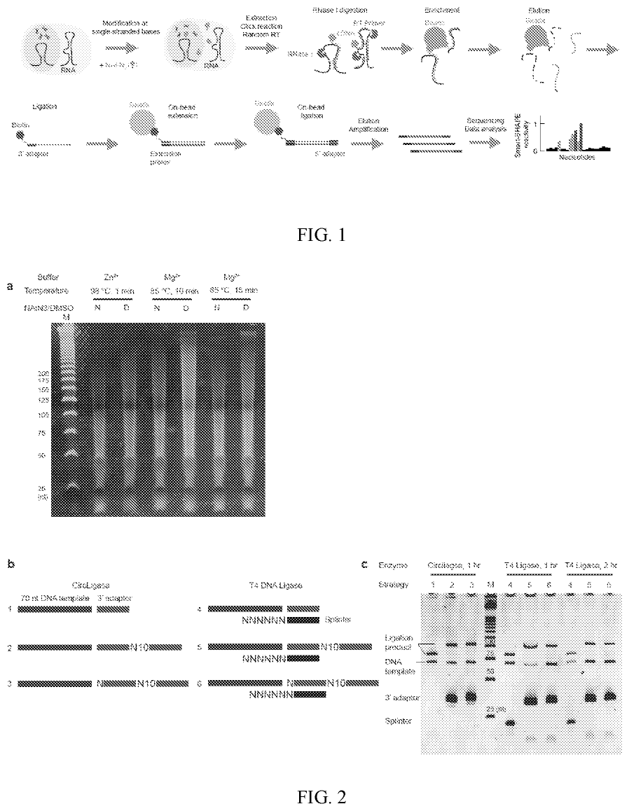

To minimize the loss of input material, we developed smartSHAPE, which combines random-primed reverse transcription, on-beads reactions, and single-stranded DNA library construction (see FIG. 1). A mixture of random primers and oligo dT was used to ensure unbiased coverage by reverse transcription. In icSHAPE, Zn²⁺ was used for RNA fragmentation before library construction, while in smartSHAPE, we used Mg²⁺ in the reverse transcription system for weak fragmentation. Compared to harsh fragmentation by Zn²⁺, weak fragmentation by Mg²⁺ not only reduced the degradation of RNA but also proceeded simultaneously with the primer annealing step, reducing one column purification step (see FIG. 2a). After random-primed reverse transcription, RNA-cDNA hybrids were subjected to RNase I digestion to remove the background signals (see below), and hybrids with modifications were enriched using streptavidin beads. Hybrids were then denatured and cDNAs were eluted and purified.

The subsequent single-stranded DNA library construction was performed with most steps on magnetic beads, and the original gel extraction and column purification steps can be replaced by simple magnetic bead washing, such that the efficiency of library construction was greatly improved, and the process was simplified. Specifically, biotinylated adapters were ligated to the 3′ end of cDNA fragments by CircLigase or T4 DNA ligase, enabling their immobilization with streptavidin beads (see FIGS. 2b and c). We observed comparable ligation efficiencies of over 50% for both CircLigase and T4 DNA ligase. After the ligation of 3′ adapters, we designed a primer complementary to the adapters, which generated the second strand by extension. Finally, 5′ adapters were ligated by T4 DNA ligase, and the eluted library with intact adapters was amplified to obtain the final sequencing library. In summary, the smartSHAPE method included only two column purification steps and no gel extraction step. As a result, smartSHAPE not only reduced the RNA input required from about 1 μg to as low as 1 ng (a 1,000-fold reduction in RNA requirement) but also shortened the processing time from 4 days to 2 days.

The specific procedures are as follows:

I. Cell Culturing

HEK293T cells were maintained in a DMEM medium with high glucose (Gibco) supplemented with 10% fetal bovine serum (FBS) and 1% penicillin-streptomycin.

II. smartSHAPE Library Preparation

1. Modification by labeling reagent NAI-N3 and RNA preparation.

RNA was modified in vivo by NAI-N3. Briefly, cells were rinsed and scraped in 1×PBS at room temperature. Cells were then pelleted and resuspended in 450 μL of 1×PBS, and the suspension was mixed with 50 μL of 1 M NAI-N3 or 50 μL of DMSO (as an untreated group). Reactants were incubated for 5 min at 37° C. with rotation and the reaction was then terminated after centrifugation at 2500 g for 1 min at 4° C. Cells were resuspended and lysed with 500 μL of Trizol (Invitrogen), and total RNAs were separated by isopropanol precipitation. Poly (A)*RNA was separated using poly-A selection (Ambion) or RiboErase (KAPA). RNA samples were incubated with 1 μL of RiboLock and 2 μL of 185 mM Dibo-Biotin for 2 h at 37° C. at 1000 r.p.m in a mixer (Eppendorf). Zymo RNA Clean & Concentrator-5 column was used for purification. 2. Reverse transcription, RNase digestion, enrichment, and 3′ adapter ligation. 3.5 μL of RT primer mixture (50 μM 5′-NNNNNN-3′, 50 μM 5′-NNWNNWNN-3′, and 6 μM 5′-TTTTTTTTVN-3′) and 3μ of 5× first strand buffer (Life Technologies) were added to 8.5 μL of biotinylated RNA sample. The samples were heated to 85° C. for 5 min and then slowly cooled to 4° C. (0.1° C. per s) for primer annealing and weak fragmentation. To RNAs with primers, 0.75 μL of RiboLock, 1 μL of 100 mM DTT, 1 μL of 5× first strand buffer, and 1.25 μL of SuperScript III (Life Technologies) were added for random RT. cDNA extension was performed at 4° C. for 2 min, 15° C. for 3 min, 25° C. for 10 min, 42° C. for 45 min, and 50° C. for 25 min. 5 μL of RNase I (Thermo Fisher Scientific), 3 μL of 10×TNF buffer, and 2 μL of H₂O were added to RT products, and the mixture was incubated for 30 min at 37° C. After cDNA extension, samples should be kept at below 37° C. to avoid denaturing conditions.

MyOne C1 magnetic beads (Invitrogen) (20 μL/sample) were prepared by washing three times with 1 mL of bead binding buffer (100 mM Tris-HCl pH 7.0, 1 M NaCl, 10 mM EDTA) and resuspending in 10 μL of bead binding buffer supplied with 1 μL of RiboLock. The product of RNase I digestion was mixed with pre-washed beads and incubated for 45 min at room temperature with rotation. After five washes with 500 μL of wash buffer (100 mM Tris pH 7.0, 4 M NaCl, 10 mM EDTA and 0.2% Tween-20) and two washes with 500 μL of 1×PBS, the magnetic beads bound to the cDNA samples were resuspended with 40 μL of H₂O. cDNAs were eluted by adding 5 μL of 1 M NaOH and incubated for 15 min at 70° C. at 1000 r.p.m. in a mixer to fully digest RNAs. Samples were immediately placed on a magnet, 45 μL of cDNA eluate was moved to a new tube, and 5 μL of 1 M HCl was added. The eluate was then purified on a Zymo DNA Clean & Concentrator-5 column. After RNase I digestion, DMSO groups were incubated directly and purified with NaOH. The purified samples were mixed with 1 μL (1 U) of FastAP (Thermo Fisher Scientific), 3 μL of 10×CircLigase II (Epicentre), and 1.5 μL of MnCl₂, and incubated for 10 min at 37° C. and for 2 min at 95° C. for end repair. A ligation mixture consisting of 12 μL of 50% PEG-4000 (Sigma), 1.5 μL of CircLigase II (Epicentre), and 1 μL of 10 μM 3′ adapter (see Table 1) was added and mixed by intense vortexing. Reactants were incubated for 2 h at 60° C. and then cooled down to 4° C.

TABLE 1
3′ adapter system

| Name | Sequence 5′-3′ |
|---|---|
| 3′ adapter | 5rApp/NNNNNNNNNNAGATCGGAAG/iSp18/TEG-biotin (SEQ ID |
| | No. 1) |
| Extension | TACACTCTTTCCCTACACGACGCTCTTCCGATCT (SEQ ID No. 2) |
| primer | |
| DSA-forward | GTGTGCTCTTCC (SEQ ID No. 3) |
| strand | |
| DSA-reverse | 5rApp/GGAAGAGCACACGTCTGAACTCCAGTCAC (SEQ ID |
| strand | No. 4) |
| P5 primer | AATGATACGGCGACCACCGAGATCTACACTCTTTCCCTACACG |
| | ACGCTCTT (SEQ ID No. 5) |
| P7 primer | CAAGCAGAAGACGGCATACGAGATGTGACTGGAGTTCAGACG |
| | TGT (SEQ ID No. 6) |

The C at the 3′ end of SEQ ID No. 3 was preferably modified by dd; the TCAC at the 3′ end of SEQ ID No. 4 was optionally subjected to thio-modification; an index sequence was optionally inserted between the GAGAT and GTGAC in SEQ ID No. 6.

3. 3′ Adapter Ligation and Second Strand Synthesis

MyOne C1 magnetic beads (Invitrogen) (20 μL/sample) were prepared by washing twice with 500 μL of binding buffer (10 mM Tris-HCl pH 8.0, 1 M NaCl, 1 mM EDTA, 0.05% Tween-20, 0.5% SDS) and resuspending in 250 μL of binding buffer. The ligation products were heated for 2 min at 95° C., then immediately transferred onto ice for at least 1 min, and incubated with pre-washed magnetic beads for 20 min at room temperature with rotation. The beads were then washed once with 200 μL of wash buffer A (10 mM Tris-HCl pH 8.0, 100 mM NaCl, 1 mM EDTA, 0.05% Tween-20, 0.5% SDS) and once with 200 μL of wash buffer B (10 mM Tris-HCl pH 8.0, 100 mM NaCl, 1 mM EDTA, 0.05% Tween).

The magnetic beads were resuspended with 47 μL of a master mix consisting of 40.5 μL of H₂O, 5 μL of 10× isothermal amplification buffer (NEB), 0.5 μL of 25 mM dNTP (Thermo Fisher Scientific), and 1 μL of 100 μM extension primer. The mixture was incubated for 2 min at 65° C. in a mixer at 1000 r.p.m., cooled on ice for 1 min and transferred to a pre-cooled 15° C. mixer, and then 3 μL of Bst 2.0 DNA polymerase (NEB) was added. Extension reactants were incubated from 15° C. to 37° C. (1° C./min) and held at 37° C. for 5 min (15 s of mixing per min) at 1500 r.p.m. in a mixer. The magnetic beads were washed once with 200 μL of wash buffer A, once with 50 μL of stringency wash buffer (0.1×SSC buffer, 0.1% SDS) at 55° C. at 1500 r.p.m. in a mixer (15 s of mixing per min), and once with 200 μL of wash buffer B. The magnetic beads were resuspended in 99 μL of a master mix consisting of 86.1 μL of H₂O, 10 μL of 10× Tango buffer (Thermo Fisher Scientific), 2.5 μL of 1% Tween-20 and 0.4 μL of 25 mM dNTP and 1 μL of T4 DNA polymerase (Thermo Fisher Scientific). Reactants were incubated for 15 min at 25° C. at 1500 r.p.m. in a mixer (15 s of mixing per min). The beads were washed three times as described above.

4. 5′ Adapter Ligation and Amplification

The magnetic beads were resuspended with 98 μL of a master mix consisting of 73.5 μL of H₂O, 10 μL of 10× T4 DNA ligase buffer (Thermo Fisher Scientific), 10 μL of 50% PEG-4000 (Thermo Fisher Scientific), 2.5 μL of 1% Tween-20, and 2 μL of 100 μM double-stranded adapter (DSA) (see Table 1). The DSA was annealed by heating two complementary oligonucleotides for 10 sec at 95° C. and slowly cooling to 14° C. (0.1° C./s). After the addition of 2 μL (10 U) of T4 DNA ligase (Thermo Fisher Scientific), the ligation reactants were incubated for 1 h at 25° C. at 1500 r.p.m. in a mixer (15 s of mixing per min). The beads were washed three times as described above, then resuspended in 25 μL of elution buffer (10 mM Tris-HCl pH 8.0, 0.05% Tween-20), and incubated for 10 min at 95° C. The supernatant was collected for amplification.

Samples were amplified in 40 μL of qPCR reactants (12 μL of cDNA, 20 μL of 2× Phusion HF master mix, 0.75 μL of 10 μM P7 index primer (see Table 1), 0.75 μL of 10 μM P5 primer (see Table 1), 0.4 μL of 25× SybrGreen). The qPCR instrument was programmed as follows: 98° C. for 1 min, 98° C. for 15 s, 65° C. for 30 s, and 72° C. for 45 s. After the qPCR amplification, the samples were size-selected (>150 bp) with 6% native PAGE gel. Deep sequencing was run on HiSeq X Ten (Illumina) after quantification with Qubit (Invitrogen).

II. Computational Pipeline for smartSHAPE Score Calculation

Since most of insertion sequences were shorter than 100 nt, we used only read mate 1 for subsequent processing. The smartSHAPE sequencing data was processed using icSHAPE-pipe. The processing steps were as follows: 1) The 3′ adapter was removed by Cutadapt; 2) Duplicate reads were removed; 3) The first 10 nt were removed using Trimmomatic; 4) Clean reads were mapped to human rRNA with Bowtie2; 5) The un-mapped reads were then mapped to the human (hg38) or mouse (mm10) genome using STAR; 6) Sam files were converted into .tab files using icSHAPE-pipe sam2tab; 7) The smartSHAPE score was calculated using icSHAPE-pipe calcSHAPENoCont with parameters: —N NAI_rep1.tab, NAI_rep2.tab; -size chrNameLength.txt; -out reactivity.gTab; -ijf sjdbList.fromGTF.out.tab. The sjdbList.fromGTF.out.tab and chrNameLength.txt files were generated by STAR during genome index generation.

Basically, icSHAPE-pipe calculated genome-wide smartSHAPE scores based on a sliding window scheme with a default window size of 200 nt and a step size of 5 nt, which skipped non-coding regions and concatenated exons when defining windows. Each nucleotide was calculated 40 times and only nearby nucleotides were considered during the calculations to avoid bias caused by uneven coverage of different regions in each transcript. When 5′ of a read was aligned to a 3′ adjacent site (+1 position), the reverse transcription stop signal of each site was increased by one. Reverse transcription stop signals were normalized within each window and 90% winsorization was performed to get final scores ranging from 0 to 1. The final smartSHAPE score of each base was the average score of all windows containing the base. The smartSHAPE scores were defined as NULL if the coverage is lower than 100, which means failure to probe the structure at these sites.

IV. RNA Structure Analysis

The receiver operating characteristic (ROC) curve was generated with the Python package sklearn. In summary, given a secondary structure and a list of SHAPE scores (0-1), single-stranded bases were regarded as positive samples, and double-stranded bases were regarded as negative samples. The false positive rate (FTR) and true positive rate (TPR) could be calculated if a cutoff of SHAPE scores was used to divide all bases into positive samples and negative samples. Therefore, the ROC curve could be generated by gradually adjusting the cutoff from 0 to 1. AUC is the area under the ROC curve.

RNA structure modeling: The RNA secondary structure was modeled using the Fold program in the RNAstructure package. The smartSHAPE scores could be used as constraints, with the default slope and intercept parameters.

### Example 2: Removal of m¹A Modification-Caused Background Signals by RNase I Digestion

Biotinylated total RNAs of HEK293T modified with NAI-N3 were mixed with 3.5 μL of specific RT primer and 3 μL of 5× first strand buffer. The mixture was heated to 65° C. for 5 min and incubated on ice for 2 min. The annealed samples were mixed with 0.75 μL of RiboLock, 1 μL of 100 mM DTT, 1 μL of 5× first strand buffer, and 1.25 μL of SuperScript III (Life Technologies) and incubated for 30 min at 55° C. The RT products were divided into 5 parts, wherein one group omitted both RNase I digestion and magnetic bead enrichment and one group directly performed magnetic bead enrichment. Other groups were incubated with 10 μL, 5 μL, or 2.5 μL of RNase I, respectively in a 30 μL reaction system. Sample enrichment was performed with MyOne C1 magnetic beads, and the samples were incubated with NaOH for elution as described above. Finally, all the samples were purified with Zymo DNA Clean & Concentrator-5 column and separated by 7 M urea PAGE.

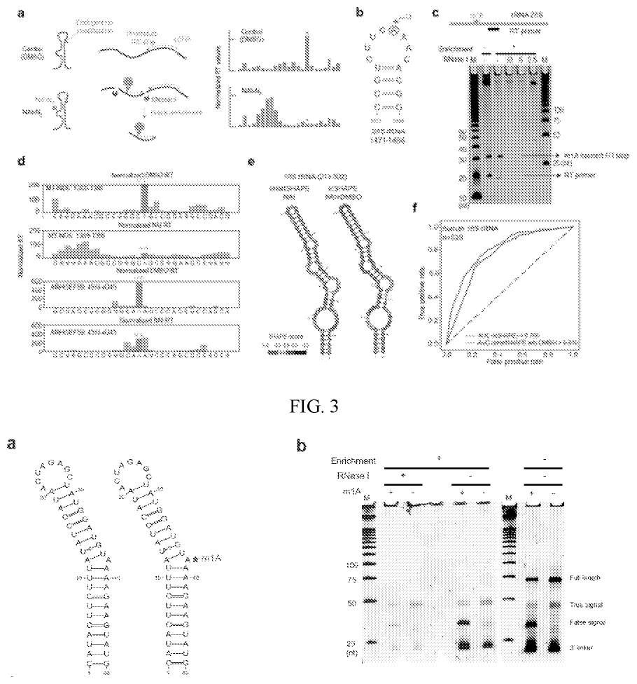

NAI-N3 in icSHAPE and smartSHAPE modifies single-stranded nucleotides and causes reverse transcription (RT) stops. However, reverse transcriptase also stops at some sites of endogenous modifications such as m¹A, local structures such as the G-quadruplexes, or simply unmodified sites by chance. These background reverse transcription stop signals will cause false positive signals in the structure score calculation. Therefore, in previous RNA structure probing methods, a DMSO control group was added to remove background signals. In smartSHAPE, however, we introduced an RNase I digestion step after reverse transcription to remove the stop signals at non-modified sites. As shown in FIG. 3a, in the process of reverse transcription, one RNA may be bound by multiple reverse transcription primers and transcribed into multiple cDNA molecules. As long as there was one modified site on an RNA, all cDNA molecules thereon could be enriched, and false signals caused by non-modified sites may be included. RNase I can specifically cleave single-stranded RNA but not RNA-cDNA hybrid strands. Therefore, RNase I digestion can cleave different cDNA molecules into separate fragments, thereby avoiding the enrichment of background signals. Theoretically, all RT signals captured in the smartSHAPE library correspond to the true modifications of the probing agent, so that the DMSO group could be omitted to further save starting materials, labor and sequencing cost.

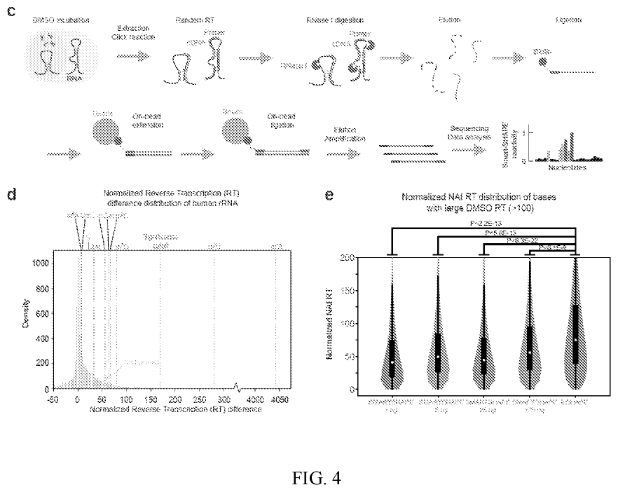

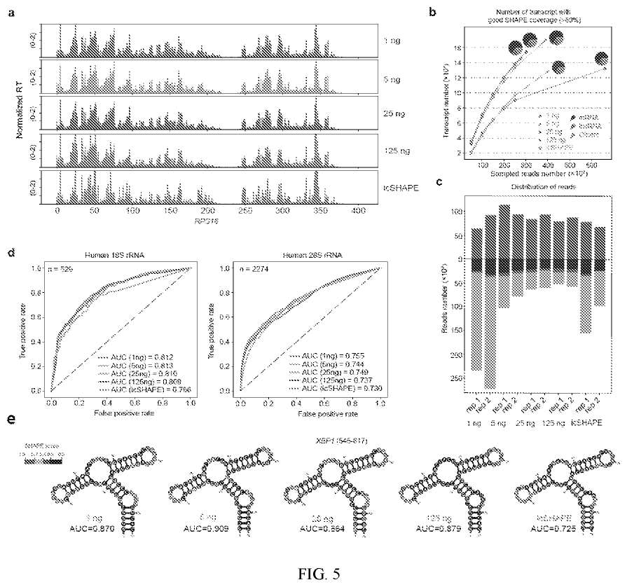

To verify that the RNase I digestion step functions as expected to remove the background reverse transcription stop signals, we designed RT primers upstream of a known m¹A modification site in human ribosomal RNA 28S (FIG. 3b). We treated HEK293T cells with NAI-N3, isolated RNA, performed Click-IT biotinylation, and then performed reverse transcription (see Example 1 for details). For samples without RNase I treatment, we observed strong background reverse transcription stop signals corresponding to the m¹A site in addition to full-length cDNA, after streptavidin magnetic bead enrichment, and the band could not be detected after RNase I digestion, which indicates that when reverse transcription was performed with NAI-N3-modified HEK293T total RNA as a template and the reverse transcription product was subjected to RNase I digestion and magnetic bead enrichment simultaneously, the background reverse transcription signals caused by m¹A modification can be effectively removed (see FIG. 3c). Importantly, the RT product associated with the m¹A site was eliminated by the RNase I treatment followed by streptavidin bead enrichment. We repeated the analysis with a synthetic RNA oligonucleotide containing an m¹A modification and observed that RT products arising from the m¹A site were also eliminated by the RNase I digestion and magnetic bead enrichment (see FIGS. 4a-b).

To further assess the removal of the background signals in smartSHAPE sequencing data, we constructed libraries from HEK293T cells treated with NAI-N3 and DMSO (see FIG. 4c). To identify the background signals, we omitted the step of RNA-cDNA hybrid streptavidin bead enrichment during the construction of DMSO libraries. Our results revealed that background signals corresponding to the known endogenous m¹A modification site could be observed in the DMSO group (see FIG. 3d). Importantly, these strong background reverse transcription stop signals were significantly reduced in the NAI-N3 libraries. Note that we observed few differences in the average number of reverse transcription stop signals between the NAI-N3 and DMSO libraries for all the other endogenous modification sites that did not induce RT stops (e.g., Am and Um), indicating that the RNase I digestion step specifically removed the background signals (FIG. 4d).

### Example 3: Performance of smartSHAPE with Different Amounts of Input RNA

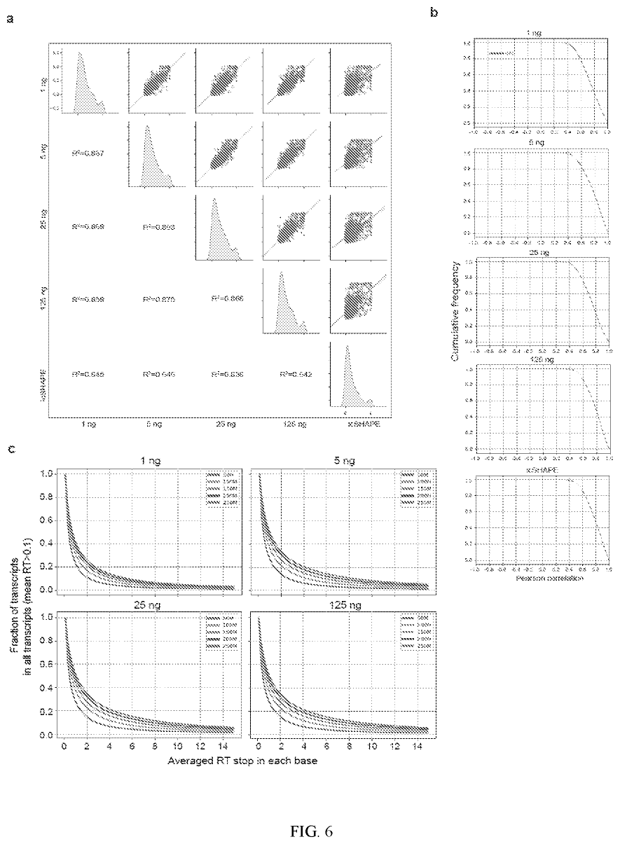

To assess the performance of smartSHAPE with different amounts of input RNA, we constructed smartSHAPE libraries by using 1 ng, 5 ng, 25 ng and 125 ng of RNA (after rRNA removal) as input to probe whole transcriptome RNA secondary structures in HEK293T cells. All smartSHAPE libraries showed good reproducibility both between libraries of different inputs (see the example in FIG. 5a and the overall statistics in FIG. 6a) and between libraries of the same input (see FIG. 6b). A transcript was defined as having “high coverage” if more than 80% of the nucleotides obtained valid smartSHAPE scores. The libraries generated with 5 ng, 25 ng and 125 ng of RNA as input successfully probed secondary structures of more than 12,000 transcripts with high coverage at a sequencing depth of 250 M, where more than 75% of the transcripts were mRNAs and lncRNAs. The number of transcripts probed by 5 ng, 25 ng and 125 ng smartSHAPE libraries was much higher than that of icSHAPE. The number of transcripts probed by the 1 ng smartSHAPE library was comparable to that of icSHAPE (see FIG. 5b, from right to left: 1 ng, icSHAPE, 5 ng, 25 ng and 125 ng, with the deepest sequencing depth as a criterion). Therefore, smartSHAPE showed higher coverage than icSHAPE at the same sequencing depth in these libraries (see FIG. 5b).

To assess the complexity of each library at different sequencing depths, we randomly sampled the same number of reads from the total raw sequencing data of each library (Table 2) and calculated smartSHAPE scores accordingly. As shown in FIG. 5b, the number of transcripts with high coverage that could be probed by 5 ng, 25 ng and 125 ng libraries at a sequencing depth of more than 250 M still rapidly increased, which indicates that the libraries all had high complexity and were not saturated, and more transcript information could be obtained by increasing the sequencing depth. Furthermore, the distribution of average reverse transcription stop signals for the three libraries at different sequencing depths was very close, which indicates that an input of 5 ng of RNA was sufficient to construct a highly complex smartSHAPE library (see FIG. 5b and FIG. 6c, where the curves from bottom left to top in FIG. 6c represent 50 M to 250 M, respectively). Finally, although we did perceive a reduction in the complexity of the 1 ng RNA input library, we still obtained more than 9,000 transcripts with high coverage at the sequencing depth of 250 M, which was comparable to icSHAPE at the same sequencing depth (which requires about 500 ng of RNA as input).

TABLE 2
The number of reads corresponding to libraries with different
sequencing depths and different processing steps

| | | | | Duplicate | Reads aligned | | Reads with | Proportion |
|---|---|---|---|---|---|---|---|---|
| | | | | reads and | to rRNA, tRNA | Reads aligned to | failed | of usable |
| | | | Raw reads | short reads | and mtRNA | genome | alignment | reads |
| 1 | ng | rep 1 | 298,220,232 | 205,776,407 | 3,269,725 | 63,959,788 | 25,214,312 | 21.45% |
| | | rep2 | 364,981,941 | 235,082,383 | 4,880,690 | 92,285,593 | 32,733,275 | 25.28% |
| 5 | ng | rep 1 | 217,786,578 | 67,450,559 | 6,780,224 | 114,501,710 | 29,054,085 | 52.58% |
| | | rep 2 | 172,584,402 | 48,699,057 | 6,116,097 | 94,134,035 | 23,635,213 | 54.54% |
| 25 | ng | rep 1 | 147,995,292 | 36,285,330 | 5,623,967 | 84,178,208 | 21,907,787 | 56.88% |
| | | rep2 | 154,431,955 | 36,416,319 | 3,909,102 | 94,201,470 | 19,905,064 | 61.00% |
| 125 | ng | rep 1 | 132,277,401 | 24,995,995 | 7,554,185 | 79,560,818 | 20,166,403 | 60.15% |
| | | rep2 | 145,538,781 | 30,164,671 | 7,010,173 | 88,024,364 | 20,339,573 | 60.48% |

We further compared the proportion of usable sequencing reads in each library. Both icSHAPE and smartSHAPE used random sequence molecular tags adjacent to the 3′ adapter to mark PCR duplication. PCR duplicate reads and reads that were too short to be aligned to the genome or reads that were aligned to rRNAs were useless for calculating RNA structure scores and needed to be discarded. The remaining reads (those aligned to the genome) were defined as usable reads. We observed that more than 60% of the total sequencing reads were usable in the 5 ng, 25 ng and 125 ng libraries. In contrast, only about 40% of the reads in the icSHAPE library generated with 500 ng of RNA as input were usable, showing that the 5 ng, 25 ng and 125 ng smartSHAPE libraries had much more reads that could be aligned to the genome than the icSHAPE library (see FIG. 5c). However, only about 20% of reads were usable in the 1 ng library. Considering sequencing costs, we suggested that the smartSHAPE library construction should use more than 1 ng of RNA as input (see FIG. 5c).

To assess the accuracy of smartSHAPE, we plotted ROC curves for the modifiable bases in 18S and 28S rRNAs by using the calculated smartSHAPE values. The AUCs of different inputs of smartSHAPE library 18S exceeded 0.8, and those of 28S exceeded 0.7, indicating good concordance between the smartSHAPE data and the known structure models, and the accuracy of the smartSHAPE library being significantly higher than that of icSHAPE (see FIG. 5d). We also evaluated smartSHAPE values by using known structure elements in the human XBP1 transcripts. In fact, we observed good concordance between the smartSHAPE values and the known structure models, and the area under the curve of the smartSHAPE library was significantly higher than that of the icSHAPE library (see FIG. 5e).

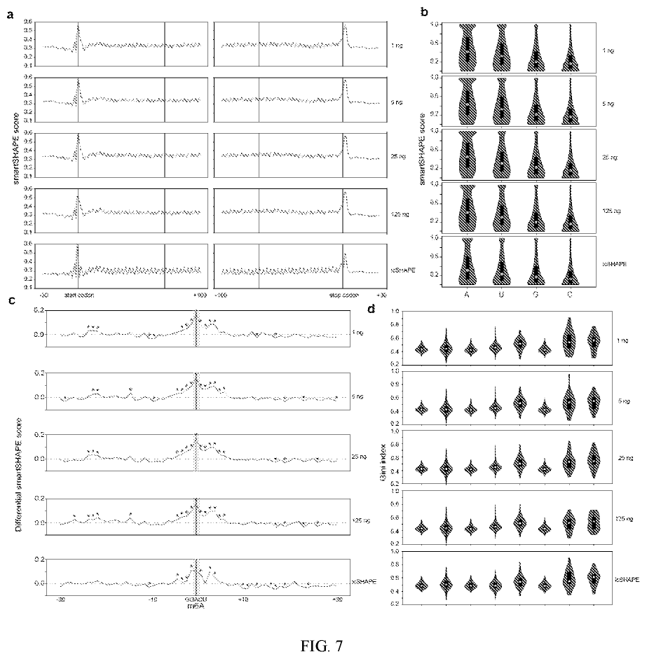

We also examined other quality control parameters of the smartSHAPE library. Similar to the previous findings, the smartSHAPE data revealed structural features at translation initiation and termination sites, as well as the 3-nucleotide periodicity in CDS regions (see FIG. 7a). Due to the generally weaker hydrogen bond of AU compared to CG base pairs, the smartSHAPE values at A and U nucleotides were higher than those at C and G nucleotides (see FIG. 7b). Compared to background regions containing the same “GGACU” motif in the smartSHAPE data, m⁶A methylated regions showed higher smartSHAPE values, which agrees with the conclusion that m⁶A regions tended to be single-stranded (see FIG. 7c). The Gini index is used to quantify how dense RNA structures are in a transcript, and a higher Gini index indicates more double-stranded RNA structures. The Gini index values of mRNAs and lncRNAs were lower than those of pseudogenes, miRNAs and snoRNAs, which agrees with previous findings (see FIG. 7d).

In summary, smartSHAPE can accurately and reliably probe RNA structures in different amounts of input samples, while requiring only a small fraction of the amount of input RNA required by other state-of-the-art in vivo RNA structure probing methods, and smartSHAPE can still accurately probes RNA structures when using a small amount, e.g., 1 ng, of RNA as input.

Therefore, smartSHAPE should be fairly suitable for many biomedical applications where the acquisition of large amounts of sample materials is extremely challenging.

### Example 4: Computational Pipeline for smartSHAPE Score Calculation

We developed a new analysis pipeline for the calculation of RNA structure scores based solely on NAI-N3 libraries (see Example 1). Briefly, smartSHAPE values were calculated by normalization and winsorization of RT stop signals in a sliding window fashion across all exons, and the smartSHAPE values for bases with coverage below 100 were defined as NULL (default window size=20 nt, step size=5 nt). We assessed the performance of the new pipeline by using a known structure model of human ribosomal RNA 18S (see Example 1). By plotting a receiver operating characteristic (ROC) curve, we observed that the smartSHAPE scores calculated with the new pipeline were better than the published icSHAPE data, and the area under the curve (AUC) of the smartSHAPE values was significantly higher than that of the icSHAPE values (see FIGS. 3e-f). These results further indicate that the RNase I digestion and streptavidin bead enrichment steps effectively removed the background signals, eliminating the need for the DMSO library as a control.

### Example 5: Whole Transcriptome Level RNA Structure Probing in Mouse Macrophages by smartSHAPE

Citrobacter rodentium was grown overnight in LB broth with shaking at 37° C. C57BL/6J mice (6-8 weeks) were infected with a total volume of 200 μL of 2×10⁹ CFUs of Citrobacter rodentium by gavage and sacrificed on day 5 post-infection. Intestinal tissue was collected and placed in ice-cold Hank's balanced salt solution (HBSS) free of calcium and magnesium. The intestine was cut open longitudinally and cut into 1.5 cm pieces and incubated twice at 37° C. for 20 min in HBSS containing 10 mM HEPES, 10 mM EDTA (Promega) and 1 mM dithiothreitol (DTT, Fermentas) to remove epithelial cells and mucus. Then the tissue was washed with HBSS containing 10 mM HEPES and digested with slow rotation at 37° C. for 75 min in RPMI 1640 (containing calcium and magnesium) containing 5% heat-inactivated fetal bovine serum (FBS), 1 mg/mL collagenase IV (Sigma), 1 mg/mL dispase (Roche), and 100 μg/mL DNase I (Sigma). The digested tissue was homogenized by vigorous shaking, passed through a 70 μm cell strainer and resuspended in 40% Percoll (GE health care) solution, and the suspension was then gradient-density centrifuged at 2,500 rpm for 20 min at room temperature. And red blood cells were lysed with ACK lysis buffer. After staining, Ly6C⁺ and Ly6C⁻ colonic macrophages were sorted on FACSAria4 laser (BD).

Innate immunity is precisely regulated to effectively eliminate pathogens while avoiding tissue damage caused by excessive immune responses. The mediators of these immune responses generally show transient expression to induce and subsequently eliminate inflammation. Post-transcriptional regulation is crucial for the rapid inhibition of protein expression of key inflammatory mediators, in which RNA structures play an important role in the regulation of RNA degradation and translation. For example, the GAIT element (the only riboswitch in mammalian cells) blocks the translation of the Vegfa gene in macrophages by recruiting GAIT complex when switching into a hairpin conformation.

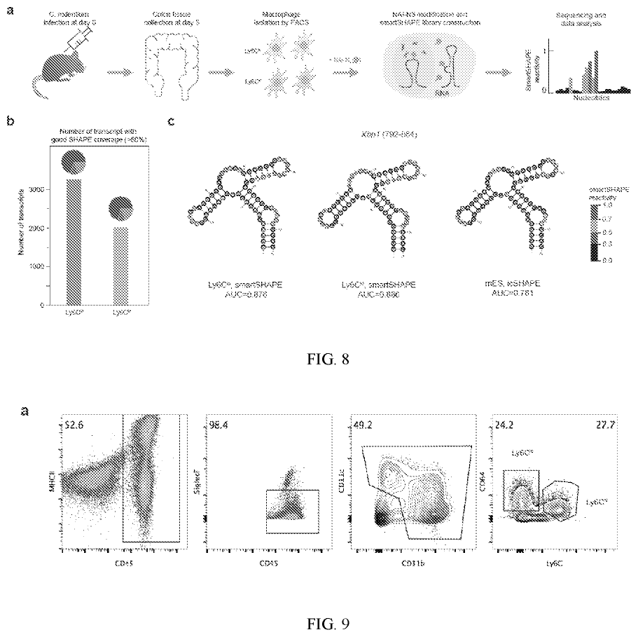

To identify new post-transcriptional regulatory RNA structure elements in immune cells, we used smartSHAPE to probe RNA secondary structure whole transcriptome in intestinal macrophages isolated from mice infected with Citrobacter rodentium (see FIG. 8a and FIG. 9a), constructed a mouse intestinal inflammation model by infecting mice with Citrobacter rodentium, and sorted Ly6C^{lo} tissue resident macrophages and Ly6C^{hi} pro-inflammatory macrophages from the intestine five days later, and finally probed RNA secondary structures in the two types of intestinal macrophages by smartSHAPE. Each mouse only had 5×10⁴ intestinal macrophages, and existing RNA structure probing methods would not work. It is noteworthy that this is the first global RNA structural data of mammalian immune cells to our knowledge.

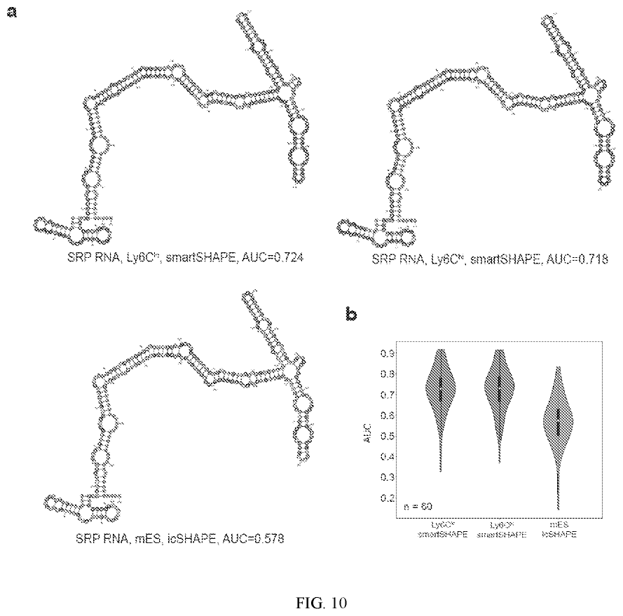

The intestinal macrophages are essential for maintaining a balance between immune responses and antigen tolerance in the intestines. Specifically, monocytes recruited from blood differentiate into Ly6C^{lo} tissue resident macrophages, which maintain intestinal homeostasis by producing anti-inflammatory cytokines such as Interleukin (IL)-10. However, during intestinal inflammation, circulating monocytes differentiate into Ly6C^{hi} pro-inflammatory macrophages, which trigger inflammation by producing pro-inflammatory cytokines such as IL6, IL1b, and IL12. To explore the potential differences in the RNA structure between tissue resident and pro-inflammatory macrophages, we used about 100 ng of total RNA to perform smartSHAPE 20 library construction for Ly6C^{lo} and Ly6C^{hi} macrophages. From the smartSHAPE data of Ly6C^{lo} and Ly6C^{hi} macrophages, we obtained the structural information of more than 3,000 and more than 2,000 transcripts with high coverage, respectively (see FIG. 8b). The smartSHAPE values of the known structure elements of the Xbp1 transcript and SRP RNA showed good agreement with known structure models and had significantly much higher AUCs compared to the icSHAPE scores (see FIG. 8c and FIG. 10a). The AUC average values of the smartSHAPE values of the two types of macrophages in a group of 60 RNAs of known structures were much higher than the AUCs of the published icSHAPE values of mouse embryonic stem cells, which indicates high smartSHAPE data quality (see FIG. 10b).

It can be seen that the results of the RNA structure probing method of the present invention can be used to assess the functional states of cells, for example, immune stress responses. Similarly, the results of the RNA structure probing method can be used to assess other functional states of cells, for example, to study the effect of RNA on early development, and the occurrence and progression of cancer.

The preferred embodiments of the present invention are described in detail above, which, however, are not intended to limit the present invention. Within the scope of the technical concept of the present invention, various simple modifications can be made to the technical solution of the present invention, all of which will fall within the protection scope of the present invention.

In addition, it should be noted that the various specific technical features described in the above specific embodiments can be combined in any suitable manner without contradiction. In order to avoid unnecessary repetition, such combinations will not be illustrated separately.

Various embodiments of the present invention can also be combined arbitrarily, and should also be regarded as the disclosure of the present invention, as long as they do not violate the idea of the present invention.

1. An RNA structure probing method, comprising: 1. obtaining an RNA-containing sample; 2. preparing a smartSHAPE library; and 3. RNA structure probing and analysis, wherein in step 2, preparing the smartSHAPE library comprises: (1) RNA modification and preparation; (2) RNA reverse transcription, removal of background reverse transcription stop signals, and cDNA enrichment.

2. The probing method according to claim 1, wherein step 2 further comprises (3) adapter ligation, second strand synthesis, and amplification.

3. The probing method according to claim 1, wherein the background reverse transcription stop signals are caused by non-RNA modification sites.

4. The probing method according to claim 1, wherein the RNA is modified with a labeling reagent.

5. The probing method according to claim 1, wherein the RNA structure is an RNA secondary structure.

6. The probing method according to claim 1, wherein the RNA is derived from the cell.

7. The probing method according to claim 1, wherein the probing method further comprises a processing step of calculating smartSHAPE scores using a computational pipeline.

8. Use of the RNA structure probing method according to claim 1, wherein the use includes assessing functional states of cells and studying the effect of RNA on early development and the development and progression of cancer according to the result of the probing method.

9. The use according to claim 8, wherein the functional states include various physiological and abnormal states.

10. The use according to claim 8, the cells include immune cells.

11. A method for assessing a functional state of a cell, wherein the assessing method comprises probing RNA structures of the cell by the probing method according to claim 1, and assessing the functional state of the cell according to the probing result.

12. The assessing method according to claim 11, wherein the functional state of the cell is cellular inflammation, injury, ischemia, immune stress state, early developmental process, infection, or cancer proliferation.

13. The probing method according to claim 4, wherein the labeling reagent is a cell membrane penetrating reagent.

14. The probing method according to claim 13, wherein the labeling reagent is dimethyl sulfate (DMS), 1-methyl-7-nitroisatoic anhydride (1M7), 2-methylnicotinic acid imidazolide-azide (NAI-N3) or kethoxal.

15. The probing method according to claim 5, wherein the RNA is a whole transcriptome level RNA.


SEQUENCE LISTING (6 sequences)

SEQ ID NO: 1 - DNA, 10 bp, Artificial Sequence
  synthetic construct
agatcggaag

SEQ ID NO: 2 - DNA, 34 bp, Artificial Sequence
  synthetic construct
tacactctttccctacacgacgctcttccgatct

SEQ ID NO: 3 - DNA, 12 bp, Artificial Sequence
  synthetic construct
gtgtgctcttcc

SEQ ID NO: 4 - DNA, 29 bp, Artificial Sequence
  synthetic construct
ggaagagcacacgtctgaactccagtcac

SEQ ID NO: 5 - DNA, 51 bp, Artificial Sequence
  synthetic construct
aatgatacggcgaccaccgagatctacactctttccctacacgacgctctt

SEQ ID NO: 6 - DNA, 45 bp, Artificial Sequence
  synthetic construct
caagcagaagacggcatacgagatgtgactggagttcagacgtgt